(19) United States
(12) Patent Application Publication
(10) Pub. No.: US 2020/0160248 A1
(43) Pub. Date: May 21, 2020

(54) SYSTEM AND METHOD FOR ASSESSING AND IMPROVING THE QUALITY OF PROFESSIONALS IN THE WORKPLACE

(71) Applicant: LAWRENCE J. HARMON, MIAMI, FL (US)

(72) Inventor: LAWRENCE J. HARMON, MIAMI, FL (US)

(21) Appl. No.: 16/687,323

(22) Filed: Nov. 18, 2019

Related U.S. Application Data
(63) Continuation-in-part of application No. 14/702,158, filed on May 1, 2015, now abandoned.

(51) Int. Cl. G06Q 10/06; A61B 5/00; G06F 21/62; A61B 5/024

(52) U.S. Cl. CPC G06Q 10/06393; A61B 5/486; A61B 5/681; A61B 5/02438; G06F 21/6254

(57) ABSTRACT

The present invention provides for a system and method for assessing and improving the quality of professionalism in the workplace. A database is instantiated with user information from a plurality of users. A rating scheme is then created from the database, comprising a plurality of ratees and raters and validated by a validator. Surveys are automatically transmitted to the raters and received upon completion for the generation of a report for each ratee, subject to further approval. The ratees are then afforded evaluations, training and feedback on the extent to which the ratee has improved. The software modules are determined by the system based on the behaviorally anchored feedback received for each ratee. A personal biometric monitoring device may further be disposed in connection with the system for performing real-time data tracking, delivery of prevention messages, and transmittal of pertinent data for further evaluations, training, and feedback.

### CLAIM OF PRIORITY

This Continuation-in-part patent application hereby makes a claim of priority to an earlier filed and currently pending U.S. non-provisional patent application having application Ser. No. 14/702,158 and a filing date of May 1, 2015, which is incorporated herewith in its entirety.

### BACKGROUND OF THE INVENTION

### Field of the Invention

This invention is directed to a system and method for assessing and improving the quality of professionals in the workplace. More particularly, the present invention is directed to a system and method for interactive user input, data mining operations, and real-time data tracking for providing an aggregate evaluation and targeted training for a professional in the workplace.

### Description of the Related Art

In the field of human resources and industrial psychology, “360-degree feedback” or multisource feedback is feedback that comes from members of an employee's immediate work circle. This feedback may come from an employee's direct subordinates, peers, or supervisors, and may also include a self-evaluation. Feedback may additionally include external sources, such as customers or suppliers, and other interested parties. The 360-degree feedback, in contrast to direct feedback from only an employee's immediate superior, provides a more holistic assessment of an employee's quality and performance in the workplace. Accordingly, 360-degree feedback may help an employee improve his or her overall work quality and performance, because it helps to evaluate several different perspectives of that employee. 360-degree feedback is traditionally conducted in the form of surveys, and has risen in popularity recently. Today, many U.S. companies employ some type of 360-degree or multisource feedback.

Despite this growing trend, the effectiveness of current 360-degree feedback systems and methods remain a major concern. For instance, the number of raters used in each category, e.g. subordinate, peer, supervisor, may affect the reliability of the feedback. The length of time that a rater has known the ratee may affect the accuracy of the evaluation. Other factors such as how the raters or ratees are selected, potential abuse in the approval process, participation rate, the presentation of information, training and coaching, the lack of anonymity, and accountability may all affect how well a 360-degree feedback system might perform. Furthermore, a lack of real-time data tracking inhibits the effectiveness of current 360-degree feedback systems, as the information ascertained from said feedback systems may not remain in focus while one is working and as a result may fail to prevent, or otherwise improve upon, the behavioral problems identified by said feedback systems. Accordingly, there is a need for a set of tested and well-defined criteria for 360-degree feedback systems and methods that can accurately and consistently assess quality within a profession. There is also a need to present this assessment in an easily interpretable manner that effectively pinpoints particular weaknesses of an employee, as well as for the targeted training related to those weaknesses. Finally, there is also need for real-time data tracking to monitor and remind an employee of said weaknesses, thus enabling said systems and method to gather more pertinent information and attempt to prevent the occurrence of said weaknesses.

### SUMMARY OF THE INVENTION

The present invention is generally directed to a system and method for assessing and improving the quality of professionals in the workplace. As such, the present invention helps a professional gain a better awareness of how he or she is perceived by others in the work place. Accordingly, the present invention involves using perceptual data, including anonymous workplace feedback and real-time biometric analysis, to help a professional improve various teamwork, leadership, anger-management, and professional skills.

In initially broad terms, a method of the present invention may comprise first instantiating a database with user information of each of a plurality of users. Accordingly, in at least one embodiment, the users share a common workplace, or are otherwise linked by a common employer or department. The user information may include user names, titles, department, contact information, length of employment, among other relevant information.

Next, a rating scheme is created for the plurality of users, comprising a plurality of ratees and raters, wherein each ratee is to be reviewed by at least one rater. In various embodiments, ratees may be able to select their raters. In other embodiments, raters may be able to select their ratees. In yet other embodiments, a user may be able to select both ratees and raters. Users may be asked to select ratees and/or raters that they work with the most. Ratees and/or raters for a user may also be recommended to the user based on user information.

The rating scheme may then be validated by at least one validator. The validator may approve, for a user, ratees or raters selected by that user. The validator may additionally be able to remove or add additional ratees and/or raters confidentially. Multiple validation steps by a plurality of validators may be used to ensure that a single validator does not abuse his or her position to purposefully create a good or bad rating for a certain user.

After validation of the rating scheme, surveys are transmitted to each of the plurality of raters of each ratee. The survey may include information related to the ratee the rater is rating. In at least one embodiment, the survey allows a rater to provide certain behaviorally-anchored feedback about a ratee. Behaviorally-anchored feedback may comprise at least a list of motivating behaviors and/or discouraging behaviors. These behaviors may be ranked on a numerical scale. The survey may also provide for comment sections via text fields for the inclusion of free-style comments about a ratee.

After the surveys are completed, they are received for data processing. Completed surveys may be checked for accuracy and completion. Incomplete surveys may be flagged or retransmitted back to a rater for completion. In other embodiments, a rater may be unable to return a complete survey until required fields of the survey are completed.

A report is then generated for each ratee based on data mining operations of the received and completed surveys. The report may aggregate statistical quantities for the information disclosed within the surveys and display numerical values of behaviors ranked by the raters as well as graphical representations thereof. The display may categorize the raters into groups such as peers, subordinates, or superiors. The report generation step may also perform anonymity editing operations, which may comprise processes or operations designed to de-identify raters. The report generation step may also identify and cluster word-themes by performing data mining operations on the completed surveys from a ratee's raters. The report generation step may also flag inappropriate comments or automatically indicate that the inappropriate comment has been deleted. Further, the report generation step may additionally flag certain data from the surveys indicative of, for instance, elevated anger-related behaviors, for possible selection of a participating ratee, as discussed herein.

The generated reports may then be approved by at least one validator, which may involve the redaction, editing of the generated reports. For instance, a validator may be able to redact flagged inappropriate comments from the report generation step. Validation by multiple validators may also be used to ensure accuracy and to prevent abuse by any one validator. A revision history logging edits by a validator may also be included during this step. Further, during the approval process, a validator may select a given ratee to act as a participating ratee for tracking using a biometric device, as discussed herein.

After approval, the approved reports may be transmitted to the ratees. As such, each ratee will receive his or her own respective report. The transmission may be in the form of a physical report, as an email, or as electronic data stored, hosted, and accessible by a ratee.

Additionally, a debriefing step may follow the transmission of the report, which involves identifying strengths and weaknesses of a ratee with a development coach. The ratee may also set development goals. The development coach may comprise at least a partially automated or computerized process, such as an interactive web platform or application. The development coach may also comprise real person interaction via a web platform, by email, phone, web conference, or other communications methods. A report of the goals may be transmitted to the ratee, as well as a validator, during this step.

Training module(s) may then be offered to each ratee based on the results disclosed in the approved reports. For example, such training module(s) may be in accordance with the ratee's numerical scores from the plurality of raters and/or the frequency of certain word-themes and/or based on the identified development goals of the ratee, as automatically analyzed by the software. The training module(s) may comprise particular lessons in text and/or video format custom tailored for the ratee, based on the ratee's practice style, professionalism, and interpersonal and communication strengths and weaknesses. As such, the ratee or professional is able to increase his or her overall awareness and thereby improve upon any weaknesses.

Follow-up surveys may be transmitted after the training period, or after the completion of the training module(s) of a ratee. In at least one embodiment, follow-up surveys may be transmitted to each ratee's plurality of raters. The follow-up surveys may be substantially similar to the initial survey that was transmitted to each ratee's raters during the survey transmission step above. The follow-up survey may comprise additional questions relating to the ratee's behavior during and throughout the training period, as disclosed in the approved reports. As may be understood, once transmitted, said follow-up surveys will be subsequently received upon completion for the performance of the remaining steps of the system and method disclosed herein.

In addition to the aforementioned steps, participating ratees may be selected to wear a biometric device for use in additional procedures. The biometric device can include any type of biometric monitor including a mobile device, a heart rate monitor, a worn device, etc., although in a preferred embodiment it includes a wrist device to be worn be a participant. The wrist device may be able to independently connect with a computer server or may be connected through an application to a mobile device, such as a smart phone. In this regard, the biometric device may comprise only the wrist device, only the mobile device and/or a combination of both devices working together to define the biometric device. In a preferred embodiment wherein it includes a wrist device, the wrist device may have a sensor disposed therein for the tracking of the participating ratee's biometric information, including, but not limited to, the participating ratee's heart rate, the variability thereof, and the movement of the participating ratee.

The wrist device, through the mobile device, may further be disposed in input-output communication with the database. Accordingly, the mobile device may transmit the participating ratee's tracked biometric information to the database for further analytical calculations, such as determining an intervention threshold based on said tracked biometric information. The intervention threshold may, for example, be used to determine instances when a participating ratee is experiencing symptoms indicative of an anger episode, or alternatively any other heightened emotional state, which may itself lead to instances of unprofessional behavior. For instance, an intervention threshold may be met in the event a participating ratee's heart rate has increased by over 40 beats-per-minute during a period of 60 seconds. Alternatively, an intervention threshold may occur where a participating ratee's heart rate variability has declined by 10% during a period of 80 seconds while the participating ratee is disposed in a state of low activity. As may be understood, the appropriate intervention threshold may vary according to the particular biometric attributes exhibited by a given participating ratee.

Upon occurrence of an ascertained intervention threshold, as tracked by the wrist device and monitored through the application disposed on the mobile device, a prevention message may be issued, either through the wrist device or the mobile device, to the participating ratee. The prevention message may, in some instances, originate from the application. Alternatively, the prevention message may originate from the database and be output to the wrist device or mobile device. The prevention message may include general information alerting the participating ratee they are at higher risk of elevated anger-related behavior and advising said participating ratee to perform at least one of a variety of tasks, such as to count to 10 or take a walk. Further, the prevention message may contain more specified information according to the information disclosed in the approved reports, such as a reminder to avoid behaving in a certain way.

After receiving a prevention message, the participating ratee may be required to provide an acknowledgement of the receipt thereof. Such acknowledgement may, for instance, comprise a manual turn off of the notification to reaffirm and reinforce the content of the prevention message. Additionally, in some embodiments, the acknowledgement may comprise a self-survey, such as an incident report, in order for the participating ratee to provide more information and context as to his or her activity and/or state of mind at the time the prevention message was received. For instance, the incident report may ask for information pertaining to the specific activity the participating ratee was performing, who the participating ratee may have been dealing with at the time, and any other pertinent information. Further, in some embodiments, the incident report may be delayed and performed at a later time.

Finally, the incident information, such as the occurrences of a threshold incident, the prevention message delivered, associated biometric information and temporal data, and acknowledgement information, may be logged and tracked for later analysis, either through the application or database. For instance, acknowledgement information comprising an incident report may be logged as a self-survey and applied to the participating ratee's generated reports. Likewise, the prevention message delivered, and the efficacy thereof, may also be recorded to the participating ratee's generated reports. Moreover, according to the biometric information and temporal data, including the day of the week and time of the occurrence of the threshold incident, the most stressful days and times for the participating ratee may also be tracked and recorded. In such instances where a trend emerges, a preemptive prevention message may be sent to the participating ratee in an attempt to prevent a threshold incident from occurring.

Accordingly, the aforementioned system and method may provide ratees and participating ratees with additional tools to monitor, assess, and improve on their work quality, performance, anger management, and professionalism in the workplace.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1A is a flowchart illustrating a method for assessing and improving the quality of professionals within the workplace in accordance with one embodiment of the present invention.

FIG. 1B is a flowchart illustrating a method for assessing and improving the quality of professionals within the workplace in accordance with another embodiment of the present invention.

FIG. 2 is a block diagram of one embodiment of a processing configuration used in the system of the present invention.

FIG. 3 is a block diagram of one embodiment of the present invention incorporated into a network system for remote access.

FIG. 4 is a block diagram of one embodiment of a processing configuration used in the system of the present invention.

FIG. 5 is a flowchart illustrating a method for assessing and improving the quality of professionals within the workplace in accordance with another embodiment of the present invention.

FIG. 6 is a flowchart illustrating a method for providing real-time data tracking and communication with a professional, in accordance with the embodiment of the present invention as depicted in FIG. 5.

Like reference numerals refer to like parts throughout the several views of the drawings.

### DETAILED DESCRIPTION OF THE PREFERRED EMBODIMENT

As schematically represented in the accompanying drawings, the present invention is generally directed to a system and method for assessing and improving the quality of professionals in the workplace. Although the present invention may most particularly be applied to professionals in high stress work environments, or in those environments where acts of unprofessionalism or anger-related behaviors are prevalent, it may be understood that the present invention may be applied to many other professions as well. Further, as may be understood herein, at least one embodiment of the present invention may be applied particularly to the medical profession, such as physicians and other healthcare professionals whom include but are not limited to those in the medical, hospital, clinic, healthcare, malpractice, quality improvement and related businesses, and the like. Accordingly, although many exemplary embodiments may particularly note application in the medical context, such embodiments, as well as any other embodiments in accordance with disclosure contained herein, are not limited to such an extent.

In at least one embodiment of the present invention, a professional may be able to compare his or her self-perception of various workplace behaviors and skills against how others perceive the professional. Moreover, the professional may be able to track his or her incidents of anger related behavior, the causes thereof, and the effectiveness of any training and prevention techniques directed thereto. Accordingly, the objective is to provide feedback that may enable the professional to feel confident and appreciated for his or her contributions, become more aware of his or her overall workplace patterns, reduce incidents of unprofessionalism, and hopefully improve upon his or her strengths and/or remedy any weaknesses.

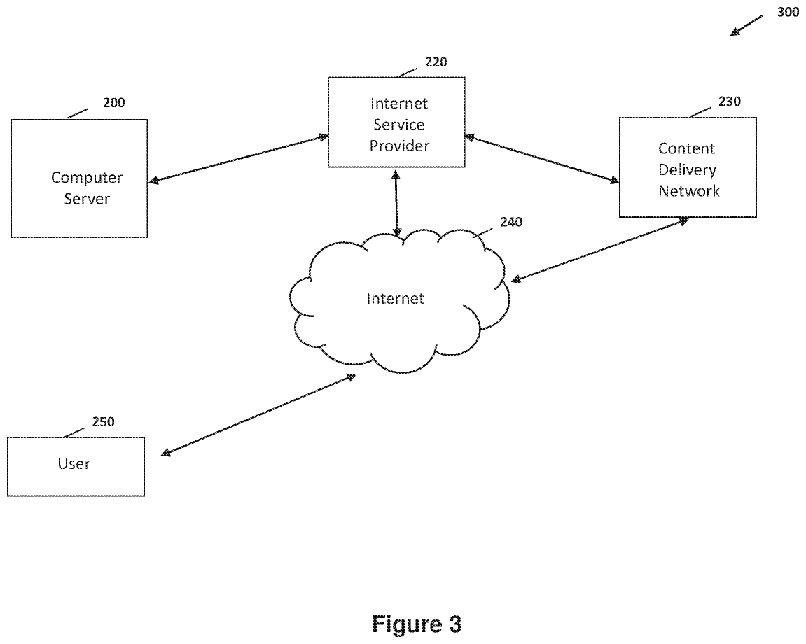

As illustrated in FIG. 3, the system 300 of the present invention may comprise a processing device or computer or computer server 200 in communications with a user 250. In at least one embodiment, the user 250 may access the computer 200 through the Internet 240 via at least one Internet service provider 220, and optionally via a content delivery network 230 which may comprise other computers or servers.

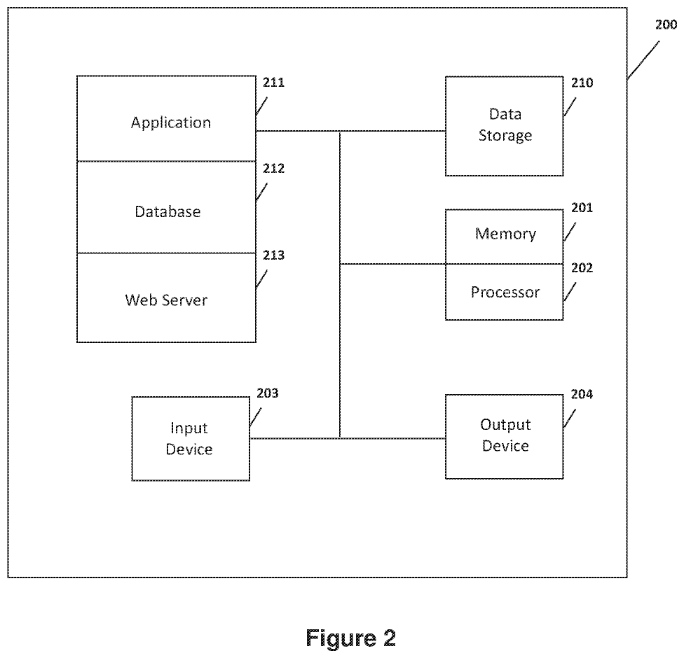

As such, and according to the schematic illustrated in FIG. 2, the processing device or computer 200 may comprise at least a memory 201 and processor 202, data storage 210, application 211, database 212, web server 213, an input device 203 and an output device 204. The memory 201 may include random access memory (RAM) or similar types of writeable memory, and may store one or more applications 211 configured to receive, store, and process information relating to assessing and improving the quality of professionals in the workplace, for execution by processor 202. Data storage 210 may include hard disks, floppy disks, CDs, DVDs, or other types of non-volatile data storage such as flash memory. The local cache, database, or data store, application, web server applications, or other data or software, for assessing and improving the quality of care provided by physicians and other healthcare professionals in the workplace, may be stored on the data storage 210. Data storage 210 may comprise multiple components stored on different computers or servers, for redundancy, security, or other reasons. Input device 203 may comprise a keyboard, mouse, cursor-control, touch-screen, infrared, microphone, digital camera, video recorder, motion or gyroscopic control, or any other device or input method appropriate for receiving, storing, and processing information related to assessing and improving the quality of professionalism in the workplace. Output device 204 may comprise any device capable and appropriate for presenting information, including but not limited to display devices, audio output devices, printers, or devices capable of transmitting information such as networking card, wireless transmitters.

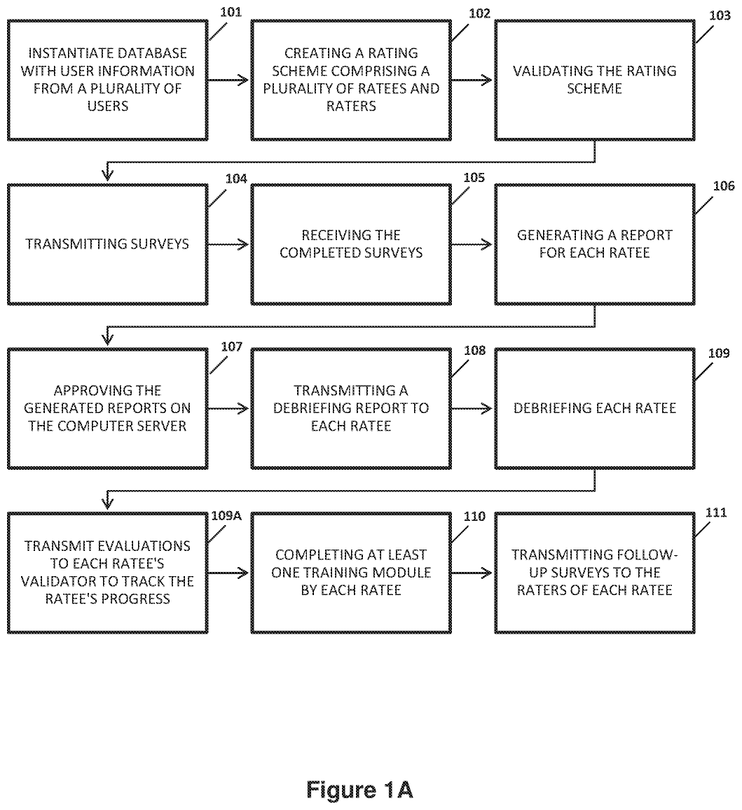

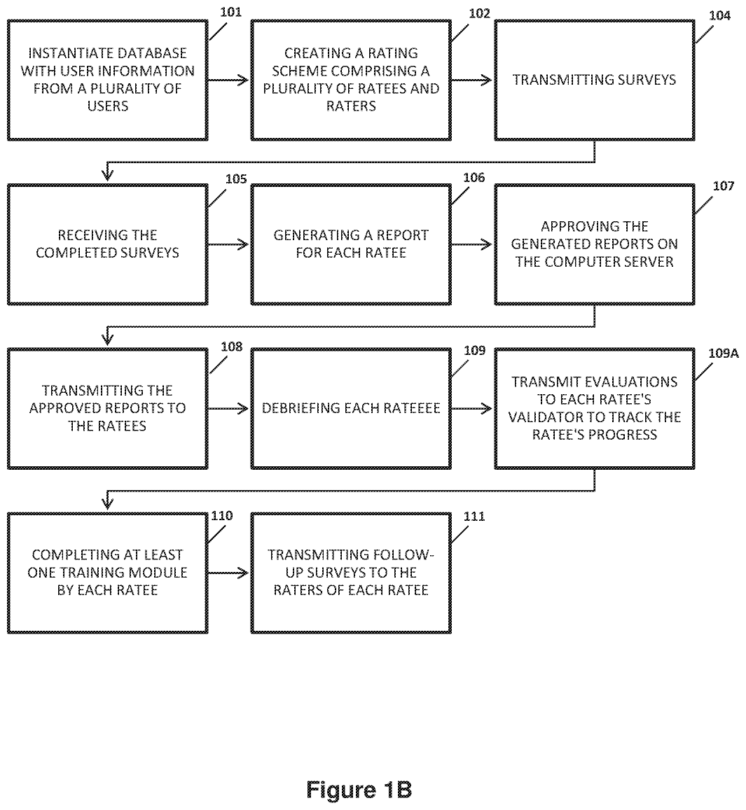

Application 211 may include web enabling components, graphic user interfaces, input and output components, components required to process and execute the steps of the method of FIGS. 1, 5, and 6 below, and the like. Applications can further provide other functions, including but not limited to production, accounting, billing, order tracking, and/or sales and marketing, and the like. Database 212 may serve as a data store for storing user information, the relationships between users, metadata, and the like. Web server 213 may provide access to applications and databases, or other information stored in memory 201 or data storage 210 to users through one or more communication networks such as the Internet. Web server 213 may allow access to a user running a web browser, co-browser, or mobile application capable of retrieving content from a network and displaying information. Examples of the processing device or computer 200 may comprise personal computers, laptop computers, tablets, mobile phones, wearable electronic devices, Internet appliances, or any processing device capable of storing data and executing software or applications. Further, as may be understood, the processing device or computer 200 may be in communication with other like processing devices or computers, as discussed by reference to FIG. 3.

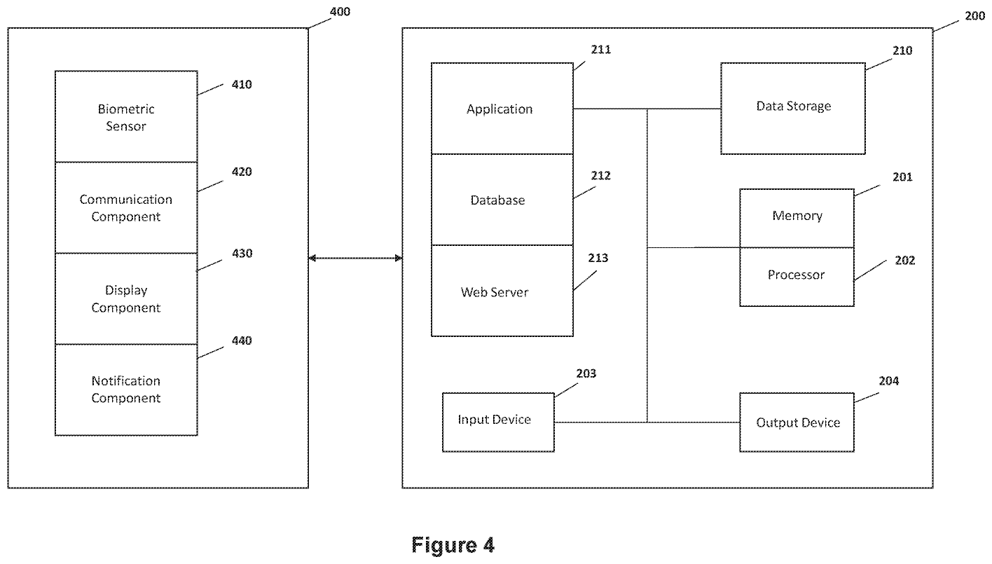

Further, as illustrated in FIG. 4, the processing device or computer 200 may be disposed in input-output relation with a wrist device 400, such as an Apple watch or any like apparatus. The wrist device 400 may be used for monitoring the biometrics of a wearer through a biometric sensor 410. Such biometrics of the wearer may include, without limitation, the heart rate, heart rate variability, movement, and/or any other relevant biometrics. The wrist device 400 may further comprise a communication component for communication with the processing device or computer 200. Such communication component may comprise wireless communication technology such as Bluetooth. Additionally, in at least one embodiment, the wrist device 400 may further comprise a display component 430, such as an LCD or OLED display, and/or a notification component 440, such as noise and/or haptic alert systems, for communication with the wearer.

In an application server embodiment, the computer or computer server 200 may comprise appropriate hardware such as a memory and at least a processor, operating system(s), software, databases, server applications, web-based applications, user interfaces or experiences (UI/UX) for performing the functionalities set forth in this application, as known to one skilled in the art. For example, the application server 213 may comprise LAMP, LYME, GLASS, LEAP, WISA, or any other solution stacks, web application frameworks, or other content management systems, and other front end and back end applications, scripts, and/or code as known to those skilled in the art.

In at least one embodiment of the present invention, the system 300 of the present invention may comprise at least one computer 200 in communication with a computer-readable medium or data storage 210 having instructions stored thereon, which when executed, cause the computer to perform operations including the steps detailed below, as depicted in the methods of FIGS. 1, 5, and 6, and any variations thereof. Accordingly, the system 300 may comprise a setup component, an enrollment or registration component, a rater selection component, validation component, a rating component, a survey instruction and transmission component, a survey reception component, a report generation component, a report approval component, a report transmission component, a debriefing and goal-setting component, a goal-reminder component, a training module component, a follow-up component. These components correspond to the method steps in FIG. 1 as detailed below. Similarly, each step of the method in FIG. 1 as detailed below may also be in the form of a code segment directed to at least one embodiment of the present invention, which is stored on a non-transitory computer readable medium, for execution by a processing device, computer, or mobile device, for assessing and improving the quality of professionalism within the workplace.

FIG. 1 depicts a flow chart of one embodiment directed to a method for assessing and improving the professionalism, teamwork, leadership, anger-management, and professionalism within the workplace. Accordingly, the method includes the steps of: instantiating a database with user information 101; creating a rating scheme 102; validating the rating scheme 103; transmitting surveys 104; receiving the completed surveys 105; generating a report for each ratee 106; approving the generated reports 107; transmitting the approved reports to the ratees 108; debriefing each ratee by using software which enables the selection of development goals and reminders in collaboration with a development coach or the ratee's leader, or on his/her own 109; transmitting evaluations to each ratee's validator or supervisor to track the ratee's progress 109A; completing when indicated by the software at least one training module by each ratee 110; and transmitting follow-up surveys to the raters of each ratee 111.

Step 101 for instantiating a database with user information includes inputting information of each of a plurality of users. The step may further include initially setting up of a database structure. The database may be stored and accessible on at least one computer locally, or remotely, such as on a computer server or cloud server. In at least one embodiment, the users share a common workplace or are otherwise linked by a common employer, department, specialty, manager, and/or occupation. The plurality of users may also include colleagues, team members, support staff, clients, patients, customers, suppliers, or other interested parties related to the system, department, specialty, employer, or workplace. Accordingly, each workplace, employer, specialty, system, or department may instantiate its own database in order to participate in the quality assessment and improvement program in a substantially tailored manner. In one embodiment, a facility liaison or a team of facility administrators may input the user information into a database. In other embodiments, each user may register and/or input his or her information, which may then be augmented and subject to further approval by an administrator. In at least one embodiment, the plurality of users instantiated in the database in step 101 comprises at least one validator who may augment and approve the final list of ratees. In other embodiments, where no validator is required, one user (rater) may select other users (ratees) and the software system will provide one survey for the rater to complete for each ratee. Each rater may be asked to select between a minimum and maximum number of ratees from several specific groups (e.g., supervisors and subordinates, physicians and nurses, etc.).

User information relating to the plurality of users may include identity information, demographic information, professional information, relational information to other users, as well as other relevant or appropriate information. Identity information may include a user's name including first and last, job title, specialty, department, and contact information. For example, in the medical context, exemplary titles in the medical profession may include whether a user is a physician, nurse, a registered nurse, a physician's assistant, an intern, a resident or other positions. Exemplary departments and/or specialties may include anesthesiology, audiology, cancer services, dentistry, emergency medicine, family medicine, medicine, neurology, neurosurgery, obstetrics and gynecology, ophthalmology, orthopedic surgery, pathology, pediatrics, pharmaceutical, psychiatry, radiation oncology, radiology, rehabilitation services, surgery, and urology. Demographic information may include age, gender, race and ethnicity. Professional information may include degrees, board certification status or other certifications, history and experience. Relational information to other users may include length of time a first user has worked with a second user and the professional relationship there between (e.g., peer, mentor, validator, facility liaison, list liaison, observer, supervisor, employee or employer, etc.).

It may be understood said aforementioned user information is merely exemplary and those skilled in the art may appreciate various informational fields may be used to instantiate a database to facilitate the review or assessment of professionals in the workplace. Those skilled in the art may also appreciate certain user information may change over time to ensure the provision of adequate professional assessment.

The input of such information may be presented as an electronic form or as part of a graphic user interface (“GUI”). A user may input such information from a computer connected to the database via a communications network such as the Internet. Alternatively, a user may input such information at the server or computer comprising the database. Certain fields may be set as required fields for the registration or enrollment of a user, whereas other fields may be set to as optional fields. A comparison may be conducted manually or via computer operations to ensure duplicate users are eliminated and user fields are accurate.

Step 102 for creating a rating scheme comprising a plurality of ratees and raters involves selecting raters and/or ratees based on the plurality of users instantiated in the database. Accordingly, this step involves the designation of users as ratees and raters, such that each designated ratee is to be rated, ranked, or reviewed by at least one rater.

In one embodiment of the present invention, ratees may be able to select their raters. Accordingly, at least one user is designated as a ratee. Next, at least one rater is designated for each ratee, wherein the rater is selected by the ratee from the plurality of users. The selection process may occur during the instantiation step as in step 101, wherein each user may register and/or input certain information into the database. Alternatively, the user may be able to access the database at a later time to select his or her raters. In certain embodiments, each user in the database may be designated as a ratee or rater by default. The validator, facility liaison and/or administrator may be omitted from the rater or ratee selection scheme, i.e. “rating scheme”. In at least one embodiment, the rater may manually add his or her own raters. A minimum and/or maximum number of raters per ratee may also be set. However, one potential pitfall of this schema might be ratees will only select raters who might rate them favorably, thus creating a biased and/or inaccurate result for various ratees.

Accordingly, in another and preferred embodiment of the present invention, raters may be able to select the ratees freely and/or subject to various criteria. The goal of this schema is to ensure a more accurate review of each ratee by placing the selection choice and power into the hands of the raters. Accordingly, at least one user is designated as a rater, or more preferably, each user in the system is designated as a rater. Next, at least one ratee is designated for each rater, wherein the ratee is selected by the rater from the plurality of users. In at least one embodiment, the rater selects from one category of users who, for example, are physicians, and from another category of users who, for example, are nurses. The selection process may occur during the instantiation step as in step 101, wherein each user may register and/or input certain information into the database. Alternatively, the user may be able to access the database at a later time to select his or her ratees. In certain embodiments, each user in the database may be designated as a rater by default. The validator, facility liaison and/or administrator may be omitted from the rating scheme in such an embodiment.

The computer server 200 and accompanying software and database may mandate selection requirements, such as requiring a minimum and/or maximum number of ratees per rater based on the category and/or perception of the rater. For example, in one embodiment each rater may be required to select at least X number of ratees from the category of physicians, and at least Y number of ratees from the category of nurses. The goal of this is to provide a two-way feedback, or team-based feedback across hierarchical barriers. In other words, by setting forth such a requirement in the rating scheme, evaluations of each ratee will not simply be limited to one's peers, but across an entire organization. In another embodiment, each rater may be required to select X number of “top performers”, Y number of “normal performers”, and Z number of “rough edges” or “poor performers”. Building such a requirement into the rating scheme may help to ensure a broader and more accurate sample of evaluations. Such a requirement is also advantageous because it allows the organization to focus on their top performers and poor performers or rough edges, for example, in the determination of who to give a bonus or other recognition, or who could benefit from various training modules. In a preferred embodiment, said rating scheme minimums, maximums, or “selection criteria” for the raters may be adjustable through the computer server 200 and accompanying database, software, and user interfaces.

In another embodiment of the present invention, users may be able to select both ratees and raters. For instance, a user may be required to select a predetermined number of raters to rate the user, as well as a predetermined number of ratees the user will rate in a subsequent survey. Minimums and/or maximums of ratees or raters, along with how many times each rater has previously completed surveys, may be configured in the database, as well as the graphic user interface, e.g. a series of forms or windows, for input of such information. In one embodiment, ratees are guided to avoid raters who have been selected too often during the prior months and, instead, to select raters who have completed relatively fewer surveys in order to spread the responsibility of completing surveys across as many raters as possible.

In at least one embodiment, users will be asked to select ratees and/or raters that they work with the most. Users may be able to pick and click other users from a list of potential raters and/or ratees. Users may be able to search for users, or to filter categories of users, e.g. by department, work unit, specialty, title, last name, first name, or a combination thereof. The system of the present invention may suggest users by certain user information. Accordingly, in at least one embodiment of the present invention, users can only select ratees and/or raters within a direct feedback group, which may be comprised mostly of individuals who have either frequent or occasional high intensity contact with the user or professional during work. In embodiments employed in the medical context this can include physician colleagues, nurses, technicians, and hospital or office staff. In at least one embodiment, an indirect feedback group may also be selected, and the indirect feedback group may be comprised mostly of individuals who are acquainted with the professional by way of others, but may not work with the professional on a daily basis. Again, in a medical context, this can include hospital administration, unit supervisors, department chiefs or chairs, medical executive committee members, medical directors, or other physicians.

Step 103 for validation of the rating scheme by at least one validator involves approving and/or adding ratees and/or raters selected by the users. Accordingly, in one embodiment of the present invention, a validator may approve, for at least one ratee, at least one rater selected by the ratee from the plurality of users. In at least one embodiment, a validator may approve, for at least one rater, at least one ratee selected by the rater from the plurality of users. Similarly, a validator may approve, for at least one user, at least one ratee or rater selected by the at least one user. In some embodiments, the validator may also be able to add and/or remove additional ratees or raters for each user. Accordingly, in various embodiments of the present invention, the validator may be able to ensure the ratees and/or raters selected by each user are actually people with a close working relationship. The validator, for example, may comprise a department or unit supervisor, a senior executive, human resources personnel, a department chief or chair, a facility liaison, or other appropriate person or persons for the validation of a rating scheme. In at least one embodiment, the validation step may be confidential, wherein the ratee or rater has knowledge of the validation step, but is not informed by the software if any users have been added or dropped and/or what users the validator adds or drops for the user. Multiple validation steps may be performed by a plurality of validators to ensure a single validator does not abuse his or her position to purposefully cause a good or bad rating for a certain user.

In certain embodiments, the validator may be able to add or designate raters and/or ratees for a user based on user information. The system of the present invention may suggest this information, for instance, through a graphical user interface. The system may also automatically designate or add raters and/or ratees for a user based on user information. For example, in at least one embodiment of the present invention, the system may use employment time and departments as parameters in designating ratees and raters. Accordingly, other users that have worked with a first user in the same department for a period of time between X and Y months may be designated as ratees and/or raters of the first user. Such a parameter setting may increase the accuracy of the rating of the first user, because the length of time may be set so other users only review a first user they have known long enough to get past the first impression, but have not known long enough that they begin to generalize the first user favorably. In at least one embodiment, a preferred period of 12 months to 36 months may be used. In other embodiments, a period of 12 months to 24 months, or a period of 12 months to 36 months may be used. Those skilled in the art may also appreciate other time periods, as well as other parameters or combination of parameters may be used to ensure accuracy of a review or assessment of a user.

Step 104 for transmitting the surveys involves transmitting a survey to each of the plurality of raters of each ratee. Each survey sent to each rater may include information related to the applicable ratee such as, for example, identification information. The survey may include other appropriate information, such as instructions. The survey may allow a rater to provide certain behaviorally-anchored feedback about a ratee. Accordingly, the survey may provide for feedback regarding predetermined motivating behaviors and discouraging behaviors, such as insight or self-awareness, communication style, leadership style, teamwork behaviors, client interactions, and time management. In embodiments employed in the medical context, such behaviors may also include, for example, clinical style patterns related to patient satisfaction and/or quality of care. In some embodiments, the survey questions may be organized in a way that correspond to the organization's codes of conduct, code of excellence, /or credentialing requirements, and/or performance or professionalism incentives. As may be understood, the survey and contents disclosed therein may vary according to the specific needs of the organization.

In one embodiment of the present invention, motivating behaviors may comprise:
1. Flexibly Adapts—adapts to change; open to suggestions
2. Manages Stress—focused and approachable under stress
3. Trustworthiness—straightforward; admits when wrong
4. Interacts Respectfully—treats others respectfully, expresses requests clearly, points out mistakes respectfully
5. Gives Information—keeps others informed; gives clear requests and explanations
6. Skillfully Communicates—expresses opinions; listens to others
7. Manages Conflict—tries to solve conflicts; handles difficult people well
8. Encourages Others—gives recognition or praise
9. Helps Out—helps out when necessary
10. Manages Time—shows up and gets things done on time
11. Decides Effectively—analyzes before deciding; makes good decisions
12. Solves Problems—identifies, solves important problems
   One or more of the above behaviors may be used in a survey in various orders. Each of these behaviors may be rated by the rater on a numerical level, such as from 1 to 5 or from 1 to 10, with 1 being the lowest and 5 or 10 being the highest. Each of the behavior categories may further comprise subcategories or more specific descriptions, which may be rated separately, e.g. “1. Flexibly Adapts” may further include “a. Is open to suggestions” and “b. Adapts to—and complies with—changing policies, procedures, and priorities.” Alternatively, only each subcategory of a category is rated separately, and the category score represents an average of the scores of all the subcategories. In at least one embodiment, a higher score for motivating behaviors of the scale would be preferred. However, as one skilled in the art may appreciate, other numerical ranges or rating methods may be used.

In one embodiment of the present invention, discouraging behaviors may comprise:
1. Passive Aggressive—criticizes indirectly; keeps only favorites informed
2. Responsibility Resistant—avoids responsibilities; responds late to calls or requests
3. Negativity—unfairly badmouths others
4. Arrogance—arrogantly demands things; talks down to others
5. Poor Anger Management—snaps at others; angry or sarcastic to others; offensive
gestures
6. Defensiveness—overreacts; defensive; blames others
7. Perfectionistic Overreaction—overreacts, insults or screams over minor problems
8. Severe Verbal Anger—yells, swears, or indicates retaliation
9. Embarrasses Others—intentionally embarrasses others publicly
10. Sexual Harassment—makes inappropriate sexual comments or gestures
11. Discriminatory Behavior—makes prejudiced comments or acts discriminatory
12. Physical Anger—bangs or throws things when angry or frustrated
   Similar to the motivating behaviors, one or more of the above discouraging behaviors may be also be used in a survey in various orders. Each of these behaviors may be rated by the rater on a numerical level, such as from 1 to 5 or from 1 to 10, with 1 being the lowest and 5 or 10 being the highest. Each of the behavior categories may further comprise subcategories or more specific descriptions, which may be rated separately, e.g. “1. Passive Aggressive” may further include “a. Criticizes certain team members, behind their back” and “b. Selectively informs only favorites of important information.” Alternatively, only each subcategory of a category is rated separately, and the category score represents an average of the scores of all the subcategories. In at least one embodiment, a lower score for discouraging behaviors of the scale would be preferred. However, as one skilled in the art may appreciate, other numerical ranges or rating methods may be used.

It should be understood motivating behaviors may include any other behaviors or identifiers indicative of motivation, encouragement, or general positive conduct towards staff, team members, coworkers, and any other relevant persons with whom the ratee associates with in a professional context. Similarly, discouraging behaviors may include any other behaviors or identifiers that indicate disruption or discouragement to others from doing their best work while around the professional. In some embodiments of the survey, other behaviors may be added as they relate to various domains identified by the client. In other embodiments of the survey, the behaviors are associated with behaviors important to the practice of a particular occupation, such as a medical specialty.

The survey transmitted to a rater may further comprise fields for free-style comments about a ratee. These fields may be titled accordingly, such as behavior the rater would like the ratee to start doing, to stop doing, and/or to keep doing. The fields may be general and may allow a rater to provide any additional feedback for the ratee, whether it's positive, negative, or neutral.

Further, step 104 may optionally involve the transmission of a survey or self-survey to each ratee. In at least one embodiment, the self-survey transmitted to the ratee is substantially the same as the survey sent to each of the plurality of raters of each ratee. Accordingly, the survey sent to the ratee or the self-survey may allow a ratee to compare how he or she perceives him or herself, compared to the perception of others. The system of the present invention may optionally send or transmit reminders at predetermined intervals to each of the raters for each of the surveys, as well as the ratee for a self-survey, until a completed survey is received back by the system or the deadline date has been reached, whichever comes first.

Step 105 for receiving the completed surveys involves receiving the completed surveys back from each of the plurality of raters, for each ratee. In some embodiments, the system may check for completion of the survey, and if a survey is incomplete the system may return an error message to the rater and request the completion of the survey. In some embodiments, a user who is a rater may be able to complete a survey online such as through a website or mobile application. In these embodiments, a user may be able to save a partially completed survey. A user may be able to submit a survey upon completion, and the system or a server and applications thereon may check for accuracy and completion. If a survey is incomplete, the system may return an error message to the user, and may further highlight or direct the user to fields or parts of the survey that requires further attention.

Step 106 for generating a report for each ratee involves data mining operations of the received and completed surveys, and the presentation of various data collated from the surveys. In at least one embodiment, the reports may be generated after a predetermined time after the surveys are transmitted, as in step 104. In other embodiments, the validator or an administrator may select when to generate the reports. In one embodiment, the report for each ratee may be generated as each survey for that ratee is completed by a respective rater. In other embodiments, the reports for all the ratees may be generated upon the receipt of the last completed survey.

In at least one embodiment of the present invention, each report generated for a ratee may comprise aggregate results of how the ratee was rated by a plurality of raters. Accordingly, at least one section of the report will present to the ratee a list of behaviorally-anchored feedback. In at least one embodiment, the behaviorally-anchored feedback comprises a list of predetermined motivating behaviors or discouraging behaviors such as the list discussed above in step 104. Similarly, the behaviorally-anchored feedback may further comprise a list of other predetermined behaviors and/or domains also presented above. As such, the rating of such categories and subcategories may comprise scores ranging from a predetermined scale of 1 to 5 or 1 to 10 or other appropriate scoring ranges. The report may present the mean, average, mode, percentile or other statistical representation of the numerical data for at least one category. This statistical representation may further be presented based on the user information, such as categories or job titles of the raters. For instance, in embodiments employed in a medical context, a ratee may be presented with the average scores given to him or her by physicians compared to nurses and other healthcare staff. Similarly, a ratee may be presented with various statistical data or indicators grouped by peers, supervisors, subordinates.

One section of the generated report for a ratee may include the top behaviorally-anchored behaviors and their associated sub-categories. These categories may be listed in ascending or descending order. For instance, the top 5 motivating behaviors and the top 5 discouraging behaviors may be generated on the report. This allows a ratee to effectively set goals on what needs the most improvement, as well as an understanding of what the ratee does well and should continue to maintain. Another section of the generated report may comprise detailed scoring of each behavioral category and subcategories. The scoring may further be broken down into averages based on groups, i.e. peers, supervisors, subordinates, and also a self-rating. The system may be able to calculate above average and below average scores. These scores may be presented in graphical form such as a line graph, a bar graph, a chart, or other graphical representations. These scores may be color-coded so a ratee can quickly identify which areas or behaviors may be very favorable or potentially problematic. For instance, a subordinate group may rate the ratee poorly in “Overreacts and defensive to suggestions” while a peer group may rate the ratee favorably. This allows a ratee to not only address a certain behavior, but also to improve that behavior with respect to interactions with a particular group in the workplace.

In at least one embodiment, the system performs data mining operations of the plurality of completed surveys to identify word-themes in order to present patterns or trends to the ratee. For example, the words “listen,” “listening,” and “listener” may count as one word-theme. Accordingly, each instance of a variation of the word “listen” may add to the occurrence of the word-theme. The most frequent word-themes may be presented first to the ratee in the ratee's report. These word-themes may further comprise favorable word-themes and unfavorable word-themes. In at least one embodiment, word-themes are presented to the ratee, categorized as behavior others would like the ratee to start, to stop, or to keep performing. These categories may be organized according to the particular fields of the surveys transmitted to the raters, as in step 104. For instance, in step 104 a rater may be prompted with a comment box asking the rater to comment on behaviors the rater would like the ratee to begin or to start performing, comments in this box will be presented to the ratee under a section with the heading “OTHERS would like you to START.” In other embodiments, these categories may be extrapolated based on the language of the feedback itself. In at least one embodiment, if a comment by a rater fits into more than one word theme, the comment may be displayed also in the second word theme area, and may further be annotated that it has already been generated earlier. Additionally, each section may also present the ratee with comments from the survey completed by the ratee, or the self-survey.

By way of example, a ratee may be presented with the following sections in a report:
YOU wrote that you would like to START:
   Delegating more effectively.
OTHERS would like you to START:
   1. Listening and taking to heart some opposing opinions.
   2. Listening to my concerns patiently.
   3. Listening to others opinions.
   4. Listening to others' input more calmly.
   5. Being as patient as you can be with the support staff when needing things in a hurry.
   6. Being patient with staff.
   7. [Listening to my concerns patiently.] (Copy of #2)
YOU wrote that you would like to STOP:
   Working more than 12 hours a day, 6 days a week, and being exhausted.
OTHERS would like you to STOP:
   1. Snapping at others sometimes when you are stressed; it can be intimidating.
   2. Snapping at those around you; it discourages our work.
   3. Choosing to snap at others in frustration.
   4. Making some snap judgments.
   5. Getting easily frustrated.
   6. Being the type of person who frustrates easily.
   7. [Choosing to snap at others in frustration.] (Copy of #3)
YOU wrote that you would like to KEEP:
   Maintaining high quality work products.
OTHERS would like you to KEEP:
   1. Being always punctual.
   2. Being tremendously punctual with your schedule; thank you.
   3. Having a great punctuality with regard to assignments.
   4. Having a high quality work product.
   5. Prioritizing quality.
      The above comments include a self-comment from the self-survey, and comments from others that may have come from a number of raters, wherein each of the raters filled in comments for the survey of the ratee. Accordingly, the word-themes are clustered or grouped together according to behaviors the raters would like the ratee to begin, stop, or keep performing, and subsequently presented to the ratee. In the example embodiment, the word-theme language is in bold, and the most frequently commented on word-theme appears first to the ratee under each section, i.e. START, STOP, and KEEP. One or more of these sections may be used in the report. Number 7 under the START section is a copy of number 2, and accordingly, the system may annotate that comment with “Copy of #2.” The word-themes may be predetermined in at least one embodiment of the present invention. For instance, the system may perform data mining operations and test the language of each of the surveys against a dictionary file, list, or database comprising a list of word-themes. Word-themes may vary by profession, profession, department, by the section of the survey, by language, or by any other distinguishing aspects. Accordingly, different dictionary files, lists, or databases of word-themes may be used depending on these and other factors.

In at least one embodiment of the present invention, the invention performs “reliability safeguard” protocols during the report generation step of step 106. These protocols are programmed into the report generation step using appropriate scripting and/or programming languages known to those skilled in the art. Specifically, one novel element of the reliability safeguard component is that it is configured to automatically hide scores from evaluations, if only a very limited number of raters have scored a ratee in a particular area. For example, a rules-based engine or programmable logic may first determine if rater number threshold is <X and, if so, then the reliability safeguard component may automatically replace the score with a notice “limited data” on the report.

Another novel aspect of the reliability safeguard component is the ability to merge several groups of raters into a single group so the aggregate scores will become more meaningful and the anonymity of the raters will be more likely to be preserved. For example, if a database schema of the computer server 200 comprises both “operating room nurses” and “floor nurses”, and a ratee's evaluation comprises “operating room nurses”<X and/or “floor nurses”<Y, then the reliability safeguard component may be configured to merge the “operating room nurses” and “floor nurses” into one grouping, displaying a new grouping called “operating room nurses+floor nurses” in the evaluation report and showing the aggregate scores from the combined two groups. Accordingly, the reliability safeguard component may be programmed with appropriate logic to automatically combine different groups of “physicians” or different groups of “nurses”, or “nurses” with “physicians” if no other similar groups exist in the database, when the number of raters in each different group is below a certain predefined threshold.

In at least one embodiment, the system may perform anonymity editing operations. Anonymity editing may comprise processes or operations designed to help de-identify raters. In one embodiment, the system may perform data mining operations to test language of the comments against dictionary files, lists, or databases comprising, for instance, standardizing the tense of initial verbs, re-arranging comments according to word-themes, standardizing comment punctuation, and/or identifying possibly inappropriate or obscene words or language. In the latter case, some inappropriate comments may be automatically redacted and/or displayed as inappropriate comments which have been deleted, or may be reviewed and flagged for removal by the validator or administrator during the approval step, as in step 107.

In at least one embodiment, the system creates a separate and more comprehensive narrative report comprised of more detailed and descriptive information, such as which group of raters provided the lowest or highest overall scores, which behaviors were scored in favorable or unfavorable ranges, how many behaviors were scored in favorable or unfavorable ranges, and/or a detailed set of recommendations comprising a customized improvement plan. The system performs data mining operations of the ratee's demographic information to automatically insert the name, address, and/or appropriate pronouns such as his or her when referring to the ratee's scores and recommendations. The system performs analyses on the ratee's scores on specific behaviors and then it selects specific recommendation sentences to insert into the comprehensive recommendations report. Based on the date the report is prepared, the system automatically inserts predetermined time intervals in the form of deadline dates by which each recommendation must be completed. When the ratee has had more than one survey, the system may perform data mining operations of the ratee's prior survey data and use the prior data to create subsequent follow-up comprehensive reports which display comparison information, for example, showing percentage improvement for each behaviors and performance trending graphs for each group of raters for each prior survey up to the most recent survey data.

The present invention may also leverage data collected nationally from a plurality of different organizations or like implementations. As such, at least one report of the present invention may comprise benchmarked reports for each ratee as compared to other ratees. The benchmark report may be adjustable by filtering the overall data, such as benchmarking a ratee in accordance to his or her region, specialty, institution, occupational group, education level, as well as other known and tracked data fields as described above.

Step 107 for approving the generated reports by the validator or other designated personnel involves flagging as an inappropriate comment to be deleted comments that contain unusually specific identity information and/or inappropriate comments which have no constructive merit. In one embodiment, one or more inappropriate comments may be automatically redacted and/or designated as a deleted inappropriate comment by the software prior to submission to the validator, or may otherwise be set to redact upon the validator's review and request. The validator or other designated personnel may perform redacting operations on the inappropriate comment in at least one embodiment. Upon approval by the validator, a report may then be transmitted to the ratee and/or to the designated debriefer who will deliver the report to, and review the report with, the ratee.

Step 108 for transmitting the approved reports to the ratees and/or to the debriefer involves transmitting the final and approved reports to the plurality of ratees. Accordingly, each ratee will be encouraged to receive his or her own respective report, the report being an end product of data mining operations from the plurality of completed surveys for that ratee. The report may display the total number of raters and the comparison norms used to prepare the report, and may comprise an introduction or instructions of how to review the report. The report may also comprise one or more sections generated by step 106 above relating to the scoring and word-themes from the plurality of completed surveys. The transmission may be in the form of a physical report, in the form of an email, an electronic file or data hosted and accessible by a ratee, or other means of transmission appropriate for the access and review of a report.

Step 109 for debriefing each ratee may be conducted by the ratee himself or herself or by a designated debriefer, such as the respective validator, facility liaison, mentor, or development coach. The debriefer helps the ratee in identifying strengths as well as potential improvement areas so that a ratee may be able to set development goals for any areas needing improvement. The designated receiving user or debriefer along with the ratee may use a partially automated module in the form of a web-based interactive platform which displays the ratee's comments from raters, and prompts the ratee to collaborate with the debriefer to select specific comments which would be appropriate development goals. The debriefing may also comprise real person interaction via the web platform, by email, phone, web conference, or by other methods of interaction. In at least one embodiment, the debriefer and ratee may discuss the meaningfulness of raters' feedback, the costs and benefits of making changes based on the feedback, approaches to improve on certain identified behavior patterns, and/or strategies to achieve the goals the ratee has set for him or herself. For instance, a number of START, STOP, or KEEP goals may be discussed with the debriefer directed to behavior the ratee should start doing, stop doing, or continue to perform. These goals may further be targeted towards a particular group of raters, for instance, the debriefer may discuss with the ratee the benefits of stopping aggressive behavior towards subordinates or staff, or with a particular department, or other group, based on specific feedback from that group. The ratee is asked to click on the development goals the ratee has created in the module, thereby indicating selection of these development goals. In at least one embodiment, the module also indicates the development goals will be automatically transmitted for review and approval to the ratee's validator, supervisor, human resources personnel, other designated persons responsible for or associated with the ratee, and/or to the development coach who may transmit the development goals of a ratee in turn to the validator and/or other designated person. An accompanying debriefing report may be transmitted to the ratee as well as the debriefer from the computer server 200.

In a preferred embodiment, the ratee may be directed to an interface within the software, where common word-themes are highlighted and automatically linked to selectable training modules. For example, if the word-theme for “listen” was repeatedly data mined out of evaluations for a ratee, such that the number of occurrences exceed X, then the computer server 200 and appropriate software logic may automatically link the word “listen” to a training module, whether in text, interactive, or video format, to the ratee. In a preferred embodiment, multiple levels of training modules may be offered to a ratee, such that a simpler level 1 “How to Listen Better” module may be offered to a ratee having an unfavorable “listen” word-theme occurrence of >X, while a more comprehensive level 2 “How to Listen Better” module might be offered to a ratee having an unfavorable “listen” word theme occurrence of >Y.

Step 109A is directed to automatically prompting the ratee and his/her validator or designated monitor to complete evaluations of the ratee's progress towards achieving his/her own development goals. The purpose is to continually monitor and track the progress of a ratee. In at least one embodiment, the user who is a validator, supervisor, or designated monitor is prompted first to rate the extent to which each development goal has been achieved during the prior evaluation period, which can be modified in the module, and offer any observational comments. Then, the module may automatically request the ratee to do the same, enabling both the ratee and validator or monitor to compare perspectives on progress towards achieving development goals.

Step 110 for completing at least one training module by each ratee involves transmitting or otherwise making available training module(s) to a ratee. The training module(s), in at least one embodiment, may be based on development goals set by the ratee in step 109. The training module(s) may be automatically selected based on identified areas needing improvement determined by statistically outlying scores and/or frequency of comment word-themes derived from the completed surveys from the ratee's raters. As such, the system of the present invention may comprise a repository of training modules including lessons, training videos, text, quizzes and exams, educational programming, or otherwise appropriate pedagogical tools. The training modules may be made available to a ratee through a web-based platform such as an interactive website or a mobile application. A ratee's progress in a training module may be tracked. Completion or certification status may be transmitted to the ratee, a validator, the ratee's supervisor, human resources personnel, or other designated persons associated with the ratee.

Step 111 for transmitting follow-up surveys to the raters of each ratee involves assessing the extent to which the ratee has improved. The follow-up surveys may be transmitted upon the completion of the at least one training module of 110, transmitted automatically based on predesignated intervals, and/or may be transmitted upon the request of the ratee, validator, facility liaison, or other designated persons. Accordingly, the same or a substantially similar survey may be again transmitted to a ratee's raters, as in step 104. In some embodiments, a brief improvement survey focuses on the extent to which the ratee has shown improvement. In other embodiments, the survey will be the same or substantially similar to ratee's development goals. Similarly, a same or substantially similar report may be generated as in 106. This will allow for the assessment of whether the ratee has improved upon any areas and/or has met the development goals set as in step 109. Of course, the survey may comprise additional questions or metrics that focus on the ratee's behaviors which have been more helpful and effective since the prior survey, and provides follow-up survey feedback to the ratee to reinforce improvement and provide feedback on remaining areas that need improvement.

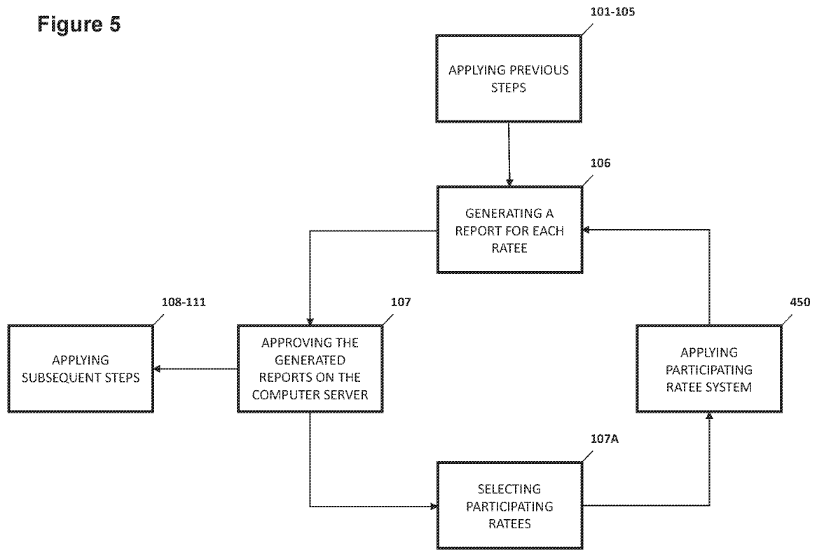

In a further embodiment of the present invention, as depicted in FIG. 5, a participating ratee system 450 may also be applied to further improve the quality of professionalism in the workplace. In such an embodiment, step 107 for approving a generated report may further include a step 107A for selecting participating ratees whom may partake in additional monitoring and training in order to obtain additional knowledge and assessment into their professionalism in the workplace.

For example, in at least one embodiment, the participating ratees may be selected by a validator or other designated personnel according to their likelihood for engaging in elevated anger-related behavior. Such likelihood may be indicated according to the results ascertained in the approved reports. For example, particular discouraging behaviors may be disposed to have a higher association with incidences of elevated anger-related behavior, hereinafter referred to “Anger Management Domain Behaviors”. For example, such Anger Management Domain Behaviors may include snapping at others when frustrated or overreacting to small mistakes. Accordingly, such behaviors may be flagged during step 106 for generating a report for each ratee and subsequently, said ratee may be selected as a participating ratee during step 107 for approving the generated reports by, for instance, the particular validator(s).

As may be understood, certain metrics may be used to effectively determine whether a given ratee should be selected as a participating ratee. For instance, while generating a report under step 106, the mean statistics for each Anger Management Domain Behavior may be calculated, by, for example, reference to other ratees disposed within the instantiated database. Subsequently, a given ratee's scores for each Anger Management Domain Behavior may be calculated and compared to the aforementioned mean statistics. In the event the ratee has scored within a specified range for a specified number of Anger Management Domain Behavior, for instance greater than two standard deviations above the mean statistics, the ratee will be flagged as a potential participating ratee. As those skilled in the art may appreciate, alternative systems may be employed for effectively selecting a participating ratee, which may depend, for instance, on the particular occupation or employer for which the system disclosed herein is applied.

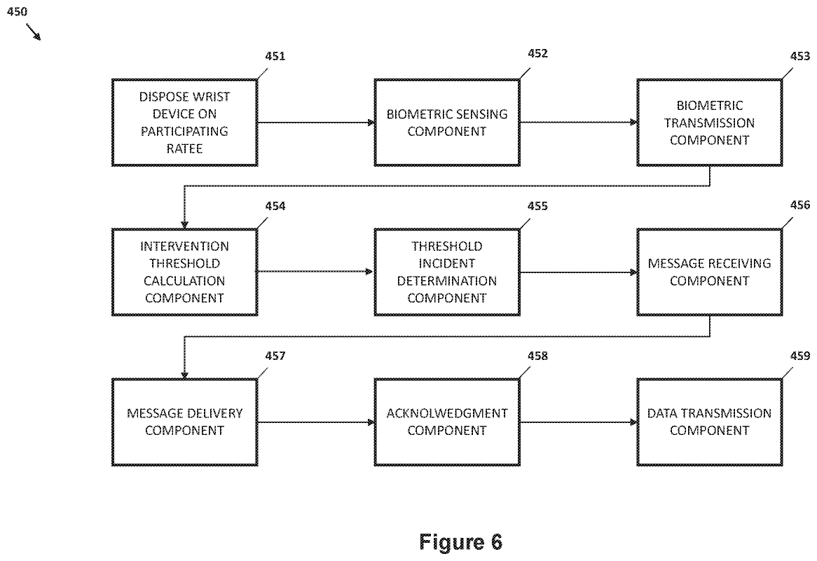

Upon selection as a participating ratee, said participating ratee may accordingly partake in step 450 for applying the participating ratee system. As can be seen in FIG. 6, in at least one embodiment the first step 451 of the participating ratee system 450 comprises disposing a wrist device on said participating ratee. As previously stated, the wrist device may utilize a biometric sensing component 452 comprising a biometric sensor 410 to track the biometrics of said participating ratee and may comprise, for example, a smart watch such as an Apple watch. Further, as previously stated, the wrist device may be connected in input-output relation to a mobile device, such as a smart phone, through a communication component 420. Further, the mobile device may include an application interconnected to both the wrist device and the database 212 of the system.

Accordingly, the wrist device may continually monitor the participating ratee's biometrics including, without limitation, the heart rate, heart rate variability, and movement. Moreover, the wrist device may optionally be set to track a participating ratee's biometrics only during his or her working hours.

Subsequent to and concurrent with the wrist device's monitoring of the participating ratee's biometrics, the wrist device may utilize a biometric transmission component 453 for output of said biometrics to the interconnected mobile device. Accordingly, the biometric information may be stored and analyzed by the application disposed on the mobile device. Particularly, the biometric information may be utilized to calculate an intervention threshold 454. The intervention threshold may be used to identify and prevent the occurrence of an anger episode wherein the likelihood of elevated anger-related behavior increases. Accordingly, by continually monitoring an individual participating ratee's biometrics, his or her typical metrics may be ascertained and used as a later comparison in determining an appropriate intervention threshold for said individual. Alternatively, certain early identification analytical metrics may be employed to determine an appropriate intervention threshold. For instance, an intervention threshold may be set to occur when a participating ratee has experienced an increase in heart rate by over 40 beats per minute during the previous 60 seconds or a decline in heart rate variability by 10% during the prior 80 seconds during a period of low activity. As may be understood, an appropriate intervention threshold may differ according to the relevant participating ratee and accordingly may be effectively changed in order to provide effective analysis.

Once an intervention threshold has been calculated, the wrist sensor may continue to monitor the biometrics of the participating ratee in order to determine when a threshold incident occurs 455. Upon such an occurrence, a prevention message may be sent, either by the application disposed on the mobile device or through the database 212 of the processing device or computer 200, and subsequently received 456 by the mobile device and/or wrist device, with the intention of alerting the participating ratee he or she is at higher risk for an episode of elevated anger-related behavior so the participating ratee may take the appropriate steps to avoid such an episode. Accordingly, subsequent to receipt of the prevention message, the wrist device or mobile device will duly deliver the prevention message 457 to the participating ratee. Said delivery may comprise, for instance, a notification alarm and/or haptic, a text message, email, or any other like notification delivery system. As may be understood, and as previously stated, the participating ratee may optionally pre-set his or her working hours, wherein a prevention message will only be delivered when said participating ratee is working. The participating ratee may also customize how frequently such prevention messages may be delivered.

In some embodiments, the prevention message may comprise a generic message relating to anger management techniques. For instance, said prevention message may include simple instructions for the participating ratee to perform, such as, without limitation:
1. Stay cool.
2. Count to 10.
3. Walk away.
4. Take a timeout.
5. Breathe deeply.
   As may be understood, such generic messages may be randomly selected by the database 212, or alternatively, the participating ratee may, upon selection as a participating ratee, select a particular prevention message he or she would like to receive, which may optionally be changed at a later point in time.

In at least one embodiment, the prevention message may alternatively be linked with the results of the participating ratee's approved report, and accordingly contain information specific to said approved report. For instance, where the approved report indicates a relevant participating ratee is prone to certain discouraging behaviors, the prevention message may include a reminder to avoid said behavior. Accordingly, if the approved report indicates the participating ratee is inclined to embarrass others, particularly when in a state of elevated anger, the prevention message may include a reminder to avoid said behavior. Further, as previously discussed with reference to FIG. 5, as the participating ratee system 450 is applied, and reports are generated 106 and subsequently approved 107, the appropriate prevention message may be altered to more appropriately address the behaviors the participating ratee is currently struggling with or seeking to improve upon. As may be understood, the efficacy of the prevention message may accordingly increase the longer the relevant participating ratee uses at least this embodiment of the present invention.

After delivery of the prevention message 457 to the participating ratee, an acknowledgement component 458 may be applied to ensure the participating ratee has received and read the prevention message. In at least one embodiment, the acknowledgement component 458 may only require the participating ratee to manually turn off the notification alert or open up the text and/or email in order to turn off any associated notification alarm and/or haptic. Alternatively, in at least one embodiment, the acknowledgment component 458 may comprise a more substantial process wherein a self-survey, such as an incident report, is additionally transmitted during the acknowledgment component 458. Optionally, the participating ratee could complete the incident report at that moment in time, or, the participating ratee could merely acknowledge the presence of said incident report and complete it at a later time, such as at the end of the day. Similar to step 105 for receiving completed surveys, as discussed above, if an incident report remains incomplete, the system may return an error message to the participating ratee, and may further highlight or direct the user to fields or parts of the incident report requiring further attention.

It may be understood such an incident report, similar to the step 104 for transmitting surveys, may comprise a variety of information specific to the participating ratee's approved reports, or alternatively, certain instantiated information 101 such as information specific to a given employer. Further, such an incident report may comprise a predetermined list of relevant incidents to report or may alternatively provide a field for free-style comments about the incident. As may be understood, the purpose of such an incident report may be to acquire additional information pertaining to the participating ratee's occurrence of a threshold incident. For instance, such relevant information may include: the work being performed, the person(s) the participating ratee was working with at that time, the presence of a specific event causing the participating ratee's heightened emotional state, the presence of outside and/or unrelated stresses in the participating ratee's personal life, or any other relevant information pertaining to said threshold incident. Additionally, the incident report may enquire about the efficacy of the prevention message in order to acquire additional information on adequate prevention measures for the particular participating ratee.

Upon the system's receipt of the participating ratee's acknowledgement, the relevant data may be transmitted 459 to the database for the generation of a report 106. Such relevant data may include, for instance, the incident information pertaining to the threshold incident. Such incident information may comprise, for example, the occurrence of a threshold incident, the prevention message delivered and the efficacy thereof, any information disclosed in the incident report, and the temporal data, such as the date and time of the threshold incident. Accordingly, such incident information may be processed, logged, and tracked in order to ascertain any additional information relevant to the participating ratee.

For instance, it may be determined there is a particular day and time a participating ratee experiences a threshold incident. In such an instance, a prevention message may optionally be preemptively sent to the participating ratee in order to attempt to prevent such an occurrence. Alternatively, it may be determined a participating ratee has reached a chronic state of high risk for anger related incidents. In such a case, a prevention message may be sent according to a predetermined time interval, such as every hour, to remind the participating ratee he or she is at a high risk for a threshold incident and to take preventative measures. As may be understood, in such instances, the requisite acknowledgement for the prevention message may be altered to more appropriately fit the context in which it is sent. For instance, where a participating ratee is deemed to be in a chronic state and is therefore receiving hourly prevention messages, the requisite acknowledgement throughout the day may only require a simple manual turn off of the notification alert with an end of the day incident report subsequently following.

As stated previously, with reference to FIG. 5, the participating ratee system may be incorporated into the survey method of FIG. 1 such that reports are generated and approved as additional information is generated from the participating ratee system. Accordingly, any such subsequent steps, such as debriefing 109, transmitting evaluations 109A, completing at least one training module 110, and transmitting follow-up surveys to the raters 111, may duly be affected by the information generated by the participating ratee system. In such a manner, the participating ratee may more effectively receive additional information and progress tracking on his or her motivating behaviors and discouraging behaviors.

Accordingly, as may be understood, application of an embodiment in accordance with the systems and methods disclosed herein may allow a professional to more accurately and efficiently monitor, assess, and improve on their work quality, performance, anger management, and professionalism in the workplace.

Any of the above steps may be completed in sequential order in at least one embodiment, though they may be completed in any other order. In at least one embodiment, the above steps may be exclusively performed, but in other embodiments, one or more steps of the steps as described may be skipped.

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A method for assessing and improving professionalism in the workplace, via an independent biometric monitoring device in communication with a computer server comprising a computer-readable medium having instructions stored thereon which when executed, cause the computer server to perform operations comprising:
(a) instantiating a database on the computer server with user information of each of a plurality of users;
(b) creating a rating scheme from the database comprising a plurality of ratees and raters, wherein each ratee is to be rated by at least one rater;
(c) transmitting surveys from the computer server for assessment of a plurality of ratees in the workplace, including:
transmitting a survey to each of the plurality of raters of each ratee;
wherein the surveys comprise at least behaviorally-anchored feedback requiring the input of numerical scores;
(d) receiving the completed surveys, including:
receiving a completed survey from at least one of the plurality of raters for each ratee;
(e) generating a report for each ratee after a pre-selected time interval based on the completed surveys for each ratee, including:
performing anonymity editing operations designed to help de-identify raters and preserve rater anonymity;
filtering and flagging inappropriate comments;
(f) approving the generated reports on the computer server, including:
preserving rater anonymity including flagging identity information from the completed surveys;
flagging inappropriate comments from the completed surveys;
performing redaction of the flagged identity information and inappropriate comments by the computer server;
selecting at least one participating ratee;
transmitting the approved reports to at least one designated receiving user;
(g) disposing the participating ratee in communication with the computer server via the biometric monitoring device:
(h) tracking biometric information of the participating ratee with the biometric monitoring device;
(i) identifying an intervention threshold for the participating ratee based upon biometric information gathered by said biometric monitoring device;
(j) determining the occurrence of the intervention threshold for the participating ratee;
(k) communicating the occurrence of the intervention threshold to the computer server;
(l) outputting a prevention message to the participating ratee based upon the determination of the intervention threshold, the prevention message comprising information specific to the participating ratee according to the approved reports.

2. The method as recited in claim 1, further comprising:
(m) receiving with the computer server an acknowledgement of the prevention message from the relevant participating ratee following transmittal of the prevention message;
(n) processing each threshold incident for the participating ratee, including:
logging incident information for the threshold incident; and
generating, approving, and transmitting at least one additional approved report for the relevant participating ratee to the at least one designated receiving user.

3. The method as recited in claim 2, wherein the acknowledgement of the prevention message comprises an incident report submitted by the risk ratee.

4. The method as recited in claim 3, further comprising:
(o) providing debriefing to each ratee, including:
identifying strengths and improvement opportunities of the ratee;
creating development goals for each ratee according to the approved reports; and
(p) transmitting follow up surveys to the raters for each ratee.

5. The method as recited in claim 1, wherein the behaviorally-anchored feedback of step (c) further comprises a list of predetermined motivating behaviors and discouraging behaviors, each to be scored by the at least one of the plurality of raters.

6. The method as recited in claim 1, wherein the prevention message of step (1) further comprises information specific to the participating ratee according to the approved reports.

7. A method for assessing and improving professionalism in the workplace via a computer server comprising a computer-readable medium having instructions stored thereon which when executed and an independent biometric monitoring device in communication therewith, said method comprising:
(a) instantiating a database on the computer server with user information of each of a plurality of users;
(b) creating a rating scheme from the database comprising a plurality of ratees and raters, wherein each ratee is to be rated by at least one rater;
(c) transmitting surveys from the computer server for assessment of a plurality of ratees in the workplace, including:
transmitting a survey to each of the plurality of raters of each ratee;
wherein the surveys comprise at least behaviorally-anchored feedback requiring the input of numerical scores;
(d) receiving the completed surveys, including:
receiving a completed survey from at least one of the plurality of raters for each ratee;
receiving a completed self-survey from each ratee;
(e) generating a report for each ratee after a pre-selected time interval based on the completed surveys for each ratee, including:
performing anonymity editing operations designed to help de-identify raters and preserve rater anonymity;
filtering and flagging inappropriate comments;
(f) approving the generated reports on the computer server, including:
preserving rater anonymity including flagging identity information from the completed surveys;
flagging inappropriate comments from the completed surveys;
performing redaction of the flagged identity information and inappropriate comments by the computer server;
selecting at least one participating ratee from the plurality of ratees;
transmitting the approved reports to at least one designated receiving user;
(g) disposing the participating ratee in communication with the computer server via the biometric monitoring device;
(h) tracking biometric information of said participating ratee with the biometric monitoring device;
(i) identifying an intervention threshold for said participating ratee based upon biometric information gathered by said biometric monitoring device;
(j) determining the occurrence of the intervention threshold for said participating ratee;
(k) communicating said occurrence of the intervention threshold to said computer server;
(l) outputting a prevention message to the participating ratee based upon the determination of the intervention threshold, the prevention message comprising information specific to the participating ratee according to the approved reports;
(m) receiving with the computer server an acknowledgement of the prevention message from the participating ratee following transmittal of said prevention message;
(n) processing each threshold incident for the participating ratee, including:
logging incident information for the threshold incident; and
generating, approving, and transmitting at least one additional approved report for said participating ratee to the at least one designated receiving user.

8. The method as recited in claim 7, wherein the step of processing each threshold incident for the participating ratee further comprises determining a chronic intervention threshold for the participating ratee.

9. The method as recited in claim 8, wherein the step of disposing each participating ratee in communication with the computer server further comprises transmitting the prevention message according to a set time period.

10. The method as recited in claim 7, wherein the behaviorally-anchored feedback of step (c) further comprises a least a list of predetermined motivating behaviors and discouraging behaviors, each to be scored by the at least one of a plurality of raters.

11. The method as recited in claim 10, wherein the behaviorally-anchored feedback of step (c) further comprises a text field.

12. The method as recited in claim 11, wherein said step of acknowledging the prevention message further comprises an incident report submitted by the participating ratee.

13. The method as recited in claim 12, further comprising:
(o) providing debriefing to the participating ratee, including:
identifying strengths and improvement opportunities of the participating ratee;
creating development goals for the participating ratee according to the approved reports; and
(p) transmitting follow up surveys to the raters for the participating ratee.

14. A system for assessing and improving professionalism in the workplace comprising:
(a) a computer system in connection with a memory, the computer system comprising:
a setup component for instantiating a database with user information for each of a plurality of users;
a rating component for creating a rating scheme from the database comprising a plurality of ratees to be rated by at least one of a plurality of raters;
a validation component for validating said rating scheme by at least one validator;
a survey transmission component for transmitting surveys comprising behaviorally-anchored feedback to said at least one of a plurality of raters;
a survey reception component for receiving the completed surveys;
a report generation component for generating a report for each ratee;
a report approval component for approving the generated reports and selecting participating ratees from the said plurality of ratees;
a report transmission component for transmitting the approved reports to at least one designated receiving user;
(b) a biometric monitoring device disposed in communicating relation with the computer system, said biometric monitoring device comprising:
a sensing component for tracking at least biometric information for each participating ratee;
a biometric transmission component for transmitting the biometric information to said computer system;
a threshold calculation component for calculating an intervention threshold for each participating ratee based on said biometric information;
a threshold determination component for determining a threshold incident upon occurrence of said intervention threshold for a particular one of said participating ratee;
a message receiving component for receiving a prevention message;
a message delivery component for delivering said prevention message to said particular participating ratee;
an acknowledgement component for receiving an acknowledgment from said particular participating ratee; and
a data transmission component for transmitting at least incident information for said particular participating ratee to said computer system.

15. The system of claim 14, wherein said report generation component is further structured to perform anonymity editing operations structured to de-identify raters and preserve rater anonymity.

16. The system of claim 15, wherein said report generation component is further structured to filter and flag inappropriate comments.

17. The system of claim 16, wherein said report approval component is further structured to perform redactions to said report according to said anonymity editing operations and said filtering and flagging of inappropriate comments.

18. The system of claim 14, wherein said behaviorally-anchored feedback of said survey transmission component further comprises at least a list of predetermined motivating behaviors and discouraging behaviors, each to be scored by said at least one of a plurality of raters.

19. The system of claim 14, wherein said prevention message of said message receiving component further comprises information specific to said relevant participating ratee according to said approved reports.

20. The system of claim 14 wherein said biometric monitoring device comprises a wrist device.

21. A system for assessing and improving professionalism in the workplace comprising:
(a) a computer system in connection with a memory, the computer system comprising:
a setup component for instantiating a database with user information for each of a plurality of users;
a rating component for creating a rating scheme from the database comprising a plurality of ratees to be rated by at least one of a plurality of raters;
a validation component for validating said rating scheme by at least one validator;
a survey transmission component for transmitting surveys comprising behaviorally-anchored feedback to said at least one of a plurality of raters;
a survey reception component for receiving the completed surveys;
a report generation component for generating a report for each ratee;
a report approval component for approving the generated reports and selecting participating ratees from the said plurality of ratees;
a report transmission component for transmitting the approved reports to at least one designated receiving user; and
(b) a personal monitoring device, said personal monitoring device structured to monitor biometric information of said participating ratee and to initiate delivery of a prevention message to said participating ratee, upon the identification of predetermined biometric parameters, and incident information to said computer system.

22. A system as recited in claim 20 wherein said personal monitoring device comprises a wrist device structured to be worn by said participating ratee.

23. A system as recited in claim 21 wherein said wrist device further comprises a heart rate monitor, said wrist device structured to initiate said prevention message upon a detection of an elevated heart rate.
